(19) United States
(12) Patent Application Publication
(10) Pub. No.: US 2021/0330494 A1
(43) Pub. Date: Oct. 28, 2021

(54) USING AN INTRAOCULAR STROBOSCOPE FOR PHACOEMULSIFIER SURGICAL DEVICE VISUALIZATION

(71) Applicant: JOHNSON & JOHNSON SURGICAL VISION, INC., Santa Ana, CA (US)

(72) Inventors: Vadim Gliner, Haifa (IL); Assaf Govari, Haifa (IL)

(21) Appl. No.: 16/859,034

(22) Filed: Apr. 27, 2020

(51) Int. Cl. A61F 9/007

(52) U.S. Cl. CPC A61F 9/00745; A61F 9/00763

(57) ABSTRACT

A device for intraocular surgery includes a handle, a needle, and a stroboscopic illumination device. The handle includes a vibrator that is configured to vibrate at a first frequency in response to a signal received from an oscillator. The needle is mechanically coupled with the vibrator and is configured to be inserted into an eye and to apply vibrations of the vibrator to the eye for emulsifying tissue. The stroboscopic illumination device is coupled with the needle and is configured to output stroboscopic light pulses at a second frequency that is set based on the first frequency, for visualizing at least a portion of the eye during emulsification by the needle.

### FIELD OF THE INVENTION

The present invention relates generally to ophthalmic procedures, and more particularly to stroboscopically illuminated ophthalmic procedures.

### BACKGROUND OF THE INVENTION

Ophthalmic procedures, such as emulsification of a cataract-clouded lens in a cataract surgery, have been in use for many years. Today, one preferred procedure for cataract removal is phacoemulsification, in which the eye's internal lens is emulsified with an ultrasonic probe and then aspirated from the eye, typically to be replaced by an artificial lens. Phacoemulsification techniques are summarized by Fine et al., in “New phacoemulsification technologies,” Journal of Cataract Refractive Surgery, June 2002, volume 28, pages 1054-1060.

The Ophthalmic operation field should typically be illuminated during surgical procedures. Methods for Illumination during Cataract surgeries are described, for example, in U.S. Pat. No. 6,786,628, which describes an instrument for providing illumination of intraocular tissue during surgery.

A vibrating piezoelectric phacoemulsification tool is typically used to disintegrate the eye's natural lens. The tool may vibrate at a relatively high frequency, and, hence, the image of the tool visualized to the surgeon may be blurry. U.S. patent application 2011/0230728 describes a method to deblur the visualized image of a vibrating tool using a stroboscopic ophthalmic illuminator.

### SUMMARY OF THE INVENTION

An embodiment of the present invention that is described herein provides a device for intraocular surgery. The device includes a handle, a needle and a stroboscopic illumination device. The handle includes a vibrator that is configured to vibrate at a first frequency in response to a signal received from an oscillator. The needle is mechanically coupled with the vibrator and is configured to be inserted into an eye and to apply vibrations of the vibrator to the eye for emulsifying tissue. The stroboscopic illumination device is coupled with the needle and is configured to output stroboscopic light pulses at a second frequency that is set based on the first frequency, for visualizing at least a portion of the eye during emulsification by the needle.

In some embodiments, the vibrator includes a piezo-electric element. In some embodiments, the stroboscopic illumination device, which is coupled with the needle for insertion into the eye, includes an optical fiber configured to receive and output the stroboscopic light pulses. In an example embodiment, the handle includes an optocoupler, which is configured to generate the stroboscopic light pulses and couple the stroboscopic light pulses with the optical fiber. In an alternative embodiment, the stroboscopic illumination device, which is coupled with the needle for insertion into the eye, includes a light source configured to generate the stroboscopic light pulses in response to an electrical signal.

There is additionally provided, in accordance with an embodiment of the present invention, a method for intraocular surgery. The method includes inserting a needle into an eye of a patient, and applying vibrations to the eye by vibrating the needle at a first frequency, so as to emulsify tissue in the eye. Stroboscopic light pulses are output, at a second frequency that is set based on the first frequency, using a stroboscopic illumination device coupled with the needle, thereby visualizing at least a portion of the eye during emulsification by the needle.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a system for intraocular surgery, in accordance with an embodiment of the present invention;

FIG. 2 is a partly pictorial, partly block diagram view of a phacoemulsification device handle and controller, in accordance with an embodiment of the present invention; and

FIG. 3 is a partly pictorial, partly block diagram view of a phacoemulsification device, in accordance with an alternative embodiment of the present invention.

### DETAILED DESCRIPTION OF EMBODIMENTS

Overview

Phacoemulsification is a cataract surgery in which the eye's internal lens is emulsified by a needle that applies ultrasonic vibration. The emulsified lens is then aspirated from the eye, typically to be replaced by an artificial lens. When the vibrating needle is observed by the surgeon, it may look blurred, due to the high frequency of the vibration. For example, if the needle vibrates along a single axis, with an amplitude of 100 um, the edge of the needle will seem smeared over a length of 200 um.

Embodiments of the present invention that are disclosed herein provide improved methods and systems for phacoemulsification. In some embodiments, a vibrating needle and a stroboscopic illumination device are integrated in the same surgical device. The needle is inserted into the eye. The stroboscopic illumination device, which is coupled with the needle, illuminates the operating field within the eye with stroboscopic light pulses. When the stroboscopic pulse frequency is suitably set, the vibrating needle is not blurred, and seems to sequence between positions in its vibration zone at a low frequency (e.g., 8 Hz); the surgeon can see the edges of the vibration by observing the edges of the sequence of needle positions.

In an embodiment, the stroboscopic illumination device comprises one or more optical fibers that receive stroboscopic light pulses from a light source (e.g., a Light Emitting Diode (LED)) that is fitted in the proximal end of the surgical device (and, thus, not inserted into the eye). In another embodiment the stroboscopic illumination device comprises the light source itself, which is coupled with electrical wires to the proximal end of the surgical device.

The disclosed surgical device is a highly effective tool for phacoemulsification. Integration of the vibrating needle and the stroboscopic illumination device in a single tool eliminates the need for the surgeon to manipulate multiple tools inside the patient's eye, and therefore simplifies the procedure and increases safety. This integration also simplifies synchronization between the frequency of the stroboscopic light pulses and the frequency of vibration of the needle.

System Description

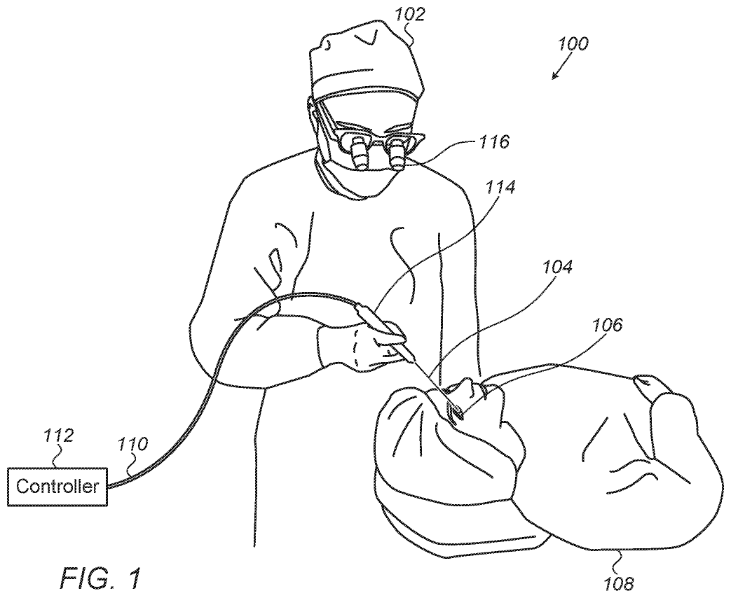

FIG. 1 is a schematic, pictorial illustration of a system 100 for intraocular surgery, in accordance with an embodiment of the present invention. The intraocular surgery may be, for example, phacoemulsification, in which a tissue in the eye, such as the natural lens, is emulsified with an ultrasonic probe and aspirated from the eye.

The figure shows a surgeon 102 manipulating a surgical device 104, the distal end of which is inserted into the lens of an eye 106 of a patient 108, e.g., through a minimal corneal or scleral incision. Device 104 comprises a handle 114 that is coupled through a flexible cable 110 to a controller (or console comprising a controller) 112. Flexible cable 110 typically comprises electrical wiring. In some embodiments, flexible cable 110 further comprises tubing, and controller 112 includes a system to pump irrigation fluid to the distal end of the surgical device and to aspirate irrigation fluid and/or lens material out of the eye via the distal end of the surgical device through the tubing. In other embodiments, cable 110 does not comprise tubing; instead, irrigation and/or aspiration tubing are coupled to handle 114 or directly to surgical device 104 and controller 112. In yet other embodiments, irrigation and/or aspiration may be done using an additional device.

Handle 114 allows the surgeon a firm grip of the surgical device during the surgery. Handle 114 may comprise a piezo-electric element or other suitable vibrator that vibrates at a frequency, typically in the ultra-sound range, in response to an electrical signal that the piezo-electric element receives from controller 112 via cable 110. The vibration of the piezo-electric element may be mechanically coupled with a needle at the distal end of surgical device 104 (not shown) causing the needle to vibrate (the end the surgical device that is inserted in the eye is referred to herein as the “distal end”). The needle, thus, vibrates at a frequency that may be blurry to a human observer.

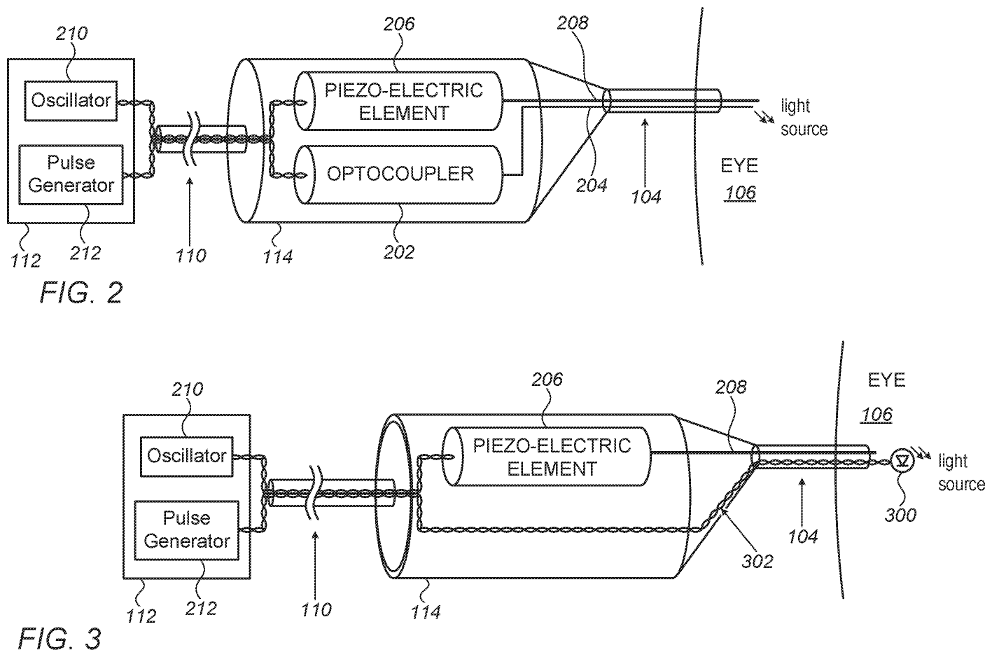

The distal end of surgical device 104 further comprises a stroboscopic illumination device, which is configured to illuminate an operating field within the eye with pulses of light (the stroboscopic illumination device is not shown in FIG. 1; it will be illustrated and further described with reference to FIGS. 2 and 3). In some embodiments, the stroboscopic illumination device comprises the end of one or more optical fibers, which run through the length of surgical device 104 and guide light pulses from a light source (e.g., a Light-Emitting Diode (LED)) that is fitted in handle 114, to the distal end of the surgical device (this embodiment will be further described with reference to FIG. 2). In other embodiments, (which will be further described with reference to FIG. 3) the stroboscopic illumination device is a light source (e.g., a LED), which is itself fitted in the distal end and is coupled to cable 110 with electrical wires that run through the length of surgical device 104. (For brevity, we will refer in the description hereinbelow to one or more optical fibers as an Optical Fiber. In various embodiments, any suitable bundling of optical fibers may be used.)

Surgeon 102 monitors the phacoemulsification process, including the vibrating needle, through magnifying glasses 116. In alternative embodiments of the present invention, surgeon 102 may use an optical microscope to visualize the phacoemulsification process.

Controller 112 sends electrical pulses to the light source, which in turn, either directly or through an optical fiber, illuminates the operating field within the eye. Typically, the pulse repetition frequency of the stroboscopic pulses is between 0.01 Hz-5 Hz, and the pulse width of each stroboscopic pulse is on the order of 40 milliseconds. Alternatively, any other suitable numerical values can be used.

In an embodiment, the frequency of the light pulses is higher or lower than the vibration frequency of the piezoelectric element, and the difference between the frequencies (referred to as the Stroboscopic Frequency) is relatively small. For example, if the entire range of motion of the needle is to be captured within 100 frames (corresponding to 100 pulses), then each frame will be offset by a phase of 2π/100. In other words, the frequency offset between the frequency of the ultrasound vibration and the frequency of the stroboscopic pulses in this example will be 1/100 of the ultrasound frequency. Thus, the vibrating needle at the distal end of surgical device 104 will be stroboscopically illuminated, allowing surgeon 102 to observe the full amplitude of the vibration with a sequence of clear images. Alternatively, any other suitable numerical values can be used.

FIG. 1 mainly shows parts that are relevant to embodiments of the present invention. Other system elements, such as irrigation and aspiration tubing, pumps and control, and their connections, as well as additional devices, are well-known in the art and are omitted for the sake of clarity.

FIG. 2 is a partly pictorial, partly block diagram view of controller 112 and handle 114 (FIG. 1), in accordance with an embodiment of the present invention. Handle 114 comprises an optocoupler 202, which is configured to convert electrical pulses into light pulses and to send the light pulses over an optical fiber 204 to surgical device 104 (FIG. 1). Handle 114 further comprises a piezo-electric element 206, which is configured to convert electrical AC current into mechanical vibrations of a needle 208, which extends from handle 114 to surgical device 104. Surgical device 104 has the form of a hollow tube, comprising needle 208 and optical fiber 204, both inserted into eye 106.

The embodiments described herein refer mainly to a piezo-electric element, but the disclosed techniques may be implemented using any other suitable kind of vibrator. The distal end of optical fiber 204, at the distal end of the surgical device, serves as the stroboscopic light source that stroboscopically illuminates the operating field, including the vibrating needle.

Piezo-electric element 206 is coupled, through cable 110, to an oscillator 210 in controller 112, and optocoupler 202 is coupled, through cable 110, to a pulse generator 212 in the controller. Oscillator 210 is configured to generate an alternating current (AC), typically at ultrasonic frequency, e.g., between 15-200 KHz, in an example embodiment around 40 KHz. The electrical coupling of oscillator 210 to piezo-electric element 206, and of pulse generator 212 to optical coupler 202, typically comprise shielded twisted pairs, to minimize radiated interference (in alternative embodiments, other suitable couplings may be used).

Generator 212 sends electrical pulses to optocoupler 202, which comprises a light-source such as a LED, optically coupled to optical fiber 204. The pulse frequency of pulse-generator 212 and the oscillation frequency of oscillator 210 are matched; the frequency difference is the stroboscopic frequency, which is typically set to a few cycles-per-second, e.g., 1/100 of the frequency of the ultrasound vibration as discussed above.

FIG. 3 is a partly pictorial, partly block diagram view of surgical device 104 (FIG. 1), in accordance with an alternative embodiment of the present invention. According to the example embodiment illustrated in FIG. 3, the stroboscopic illumination device at the distal end of surgical device 104 comprises an LED 300, which is coupled via electrical wires 302 that run through surgical device 104, handle 114 and cable 110 to pulse generator 212 in controller 112. Thus, handle 114 does not comprise an optocoupler in this embodiment.

The configurations of system 100 and its various components, illustrated in FIGS. 1 through 3, are chosen purely for the sake of conceptual clarity. In alternative embodiments of the present invention, any other suitable configurations can be used. For example, the vibrator need not necessarily be fitted in the handle. In an embodiment, oscillator 210 and pulse generator 212 are combined in a single aggregated unit. In another embodiment, an optocoupler is fitted in controller 112, which outputs an optical fiber to cable 110. In some embodiments, the stroboscopic illumination device and the vibrating needle are mounted on separate surgical devices that are inserted into the eye.

Setting the Stroboscopic Frequency

The stroboscopic frequency, defined as the difference between the needle vibration frequency and the repetition frequency of the stroboscopic light pulses, is typically set to allow an optimal view of the vibrating needle. If the stroboscopic frequency is too high (e.g., 20 Hz or higher), the surgeon will see a blurry image of the needle. If the stroboscopic frequency is too low (e.g., 1 Hz or lower), the surgical device will seem to move very slowly, and the edges of the movement range may be missed. In some embodiments, the stroboscopic frequency is preset to a suitable value (e.g., to 1/100 of a period of the ultrasound vibration as discussed above). In other embodiments, the stroboscopic frequency may be adjusted prior to or in some embodiments during, the surgery, for example, according to the preferences of the surgeon.

It will be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art.

1. A device for intraocular surgery, comprising:
a handle, comprising a vibrator that is configured to vibrate at a first frequency in response to a signal received from an oscillator; and
a needle, which is mechanically coupled to the vibrator and is configured to be inserted into an eye and to apply vibrations of the vibrator to the eye for emulsifying tissue; and
a stroboscopic illumination device, which is coupled with the needle and is configured to output stroboscopic light pulses at a second frequency that is set based on the first frequency, for visualizing at least a portion of the eye during emulsification by the needle.

2. The device according to claim 1, wherein the vibrator comprises a piezo-electric element.

3. The device according to claim 1, wherein the stroboscopic illumination device, which is coupled with the needle for insertion into the eye, comprises an optical fiber configured to receive and output the stroboscopic light pulses.

4. The device according to claim 3, wherein the handle comprises an optocoupler, which is configured to generate the stroboscopic light pulses and couple the stroboscopic light pulses with the optical fiber.

5. The device according to claim 1, wherein the stroboscopic illumination device, which is coupled with the needle for insertion into the eye, comprises a light source configured to generate the stroboscopic light pulses in response to an electrical signal.

6. A method for intraocular surgery, comprising:
inserting a needle into an eye of a patient;
applying vibrations to the eye by vibrating the needle at a first frequency, so as to emulsify tissue in the eye; and
using a stroboscopic illumination device coupled with the needle, generating stroboscopic light pulses at a second frequency that is set based on the first frequency, thereby visualizing at least a portion of the needle.

7. The method according to claim 6, wherein applying the vibrations comprises operating a piezo-electric element.

8. The method according to claim 6, wherein generating the stroboscopic light pulses comprises transmitting the stroboscopic light pulses in an optical fiber and emitting the stroboscopic light pulses from a distal end of the optical fiber.

9. The method according to claim 8, wherein generating the stroboscopic light pulses comprises generating the stroboscopic light pulses using an optocoupler and coupling the stroboscopic light pulses from the optocoupler to the optical fiber.

10. The method according to claim 6, wherein generating the stroboscopic light pulses comprises generating the stroboscopic light pulses in response to an electrical signal, by a light source coupled with the needle.
